(19) United States
(12) Patent Application Publication
(10) Pub. No.: US 2019/0032228 A1
(43) Pub. Date: Jan. 31, 2019

(54) Electrolysis System and Method for Electrochemical Ethylene Oxide Production

(71) Applicant: Siemens Aktiengesellschaft, Muenchen (DE)

(72) Inventors: Ralf Krause, Herzogenaurach (DE); Christian Reller, Minden (DE); Bernhard Schmid, Erlangen (DE); Günter Schmid, Hemhofen (DE)

(73) Assignee: Siemens Aktiengesellschaft, Muenchen (DE)

(21) Appl. No.: 16/071,780

(22) PCT Filed: Jan. 19, 2017
(86) PCT No.: PCT/EP2017/051040

§ 371 Date: Jul. 20, 2018

(30) Foreign Application Priority Data
Jan. 21, 2016 (DE) 10 2016 200 858.7

(51) Int. Cl. C25B 3/04; C25B 9/08; C25B 13/08; C25B 15/08

(52) U.S. Cl. CPC C25B 3/04; C25B 9/08; C07D 301/26; C25B 15/08; C25B 13/08

(57) ABSTRACT

An example electrolysis system for the electrochemical production of ethylene oxide includes an electrolysis cell having an anode in an anode space and a cathode in a cathode space and a gas separation element. The cathode space has a first inlet for carbon monoxide and/or carbon dioxide. The anode space is integrated into an anolyte circuit and the cathode space is integrated into a catholyte circuit. The catholyte circuit has a first product outlet for a reduction product joined to a first connecting conduit connected to the anolyte circuit. The anode space is configured for bringing a reduction product introduced via the first connecting conduit into contact with an oxidation product.

### CROSS-REFERENCE TO RELATED APPLICATIONS

This application is a U.S. National Stage Application of International Application No. PCT/EP2017/051040 filed Jan. 19, 2017, which designates the United States of America, and claims priority to DE Application No. 10 2016 200 858.7 filed Jan. 21, 2016, the contents of which are hereby incorporated by reference in their entirety.

### TECHNICAL FIELD

The present disclosure relates to electrolysis. Various embodiments may include a process and/or an electrolysis system for the electrochemical production of ethylene oxide.

### BACKGROUND

Ethylene oxide is a valuable chemical. For the commercial preparation of ethylene oxide, a silver-catalyzed gas-phase epoxidation of ethylene by means of oxygen is typically used. A process based on a catalyst which is composed of silver supported on aluminum oxide and operated at 270° C. and a pressure in the range from 1 to 10 bar goes back to the work of T. E. Lefort. In the known direct oxidation processes, there are two industrial process routes, namely that via epoxidation by means of air and that via epoxidation using pure oxygen. Because the air epoxidation process is restricted to very low conversions and also incurs some safety risks due to explosive ranges, the direct reaction with pure oxygen is carried out in a reactor under an inert gas atmosphere.

A further known process for the preparation of ethylene oxide is the chlorohydrin process in which ethylene is firstly reacted with water and chlorine to form chlorohydrin which is then dehydrochlorinated using calcium hydroxide in a second step. However, this process is no longer employed because of its high costs. In this process, chlorine is lost in the form of calcium chloride and further amounts of the external base calcium hydroxide therefore have to be introduced continuously. The demands made on the reactor materials used also represent a considerable cost outlay. In addition, from 0.1 to 0.2 metric tons of 1,2-dichloroethane and 2 metric tons of calcium chloride and 40 metric tons of contaminated water are produced as waste products for every metric ton of ethylene oxide produced by the chlorohydrin process.

Processes known hitherto for electrochemical epoxidation are anodic epoxidation at silver electrodes and, once again, the route via chlorohydrin, the dehydrochlorination of which is accompanied by various disadvantages, especially the high consumption of bases. It is consequently of industrial importance to propose an alternative and more efficient process route for electrochemical ethylene oxide production, which avoids the disadvantages described above, among others.

### SUMMARY

The teachings of the present disclosure may be embodied in an improved process and electrolysis system for ethylene oxide production. For example, some embodiments may include an electrolysis system for the electrochemical production of ethylene oxide, comprising an electrolysis cell having an anode (A) in an anode space (AR), a cathode (K) in a cathode space (KR) and at least one gas separation element (M), wherein the cathode space (KR) has a first inlet for carbon monoxide (CO) and/or carbon dioxide (CO₂) and is configured for bringing the introduced carbon monoxide (CO) and/or carbon dioxide (CO₂) into contact with the cathode (K), the anode space (AR) is integrated into an anolyte circuit (AK) and the cathode space (KR) is integrated into a catholyte circuit (KK) and the catholyte circuit (KK) has at least one first product outlet (PA1) for a reduction product which is joined to a first connecting conduit (1) which is connected to the anolyte circuit (AK) and the anode space (AR) is configured for bringing a reduction product introduced via the first connecting conduit (1) into contact with an oxidation product.

In some embodiments, there is at least one mixing unit (2) which is hydrodynamically connected to the anolyte circuit (AK) and the catholyte circuit (KK).

In some embodiments, the anode space (AR) contains bromide ions (Br⁻) and is configured for oxidizing bromide (Br⁻) to bromine (Br₂) and is also configured for taking up a reduction product, in particular ethylene (C₂H₄) transferred into the anolyte circuit (AK) and bringing it into contact with the bromine (Br₂).

In some embodiments, the gas separation element (M) comprises a diaphragm.

In some embodiments, the gas separation element (M) comprises a sulfonated polytetrafluoroethylene.

In some embodiments, there is at least one second product outlet (PA2) which is configured for taking bromine (Br₂) off from an electrolyte mixture conveyed in the anolyte circuit (AK) and/or catholyte circuit (KK) and having at least one separate reaction chamber (R) for chemical conversion back into a bromide (Br⁻), wherein the reaction chamber (R) is connected hydrodynamically via a further connecting conduit to the anode space (AR).

As another example, some embodiments may include a process for the electrochemical production of ethylene oxide by means of an electrolysis system as described above, wherein carbon monoxide (CO) and/or carbon dioxide (CO₂) are/is introduced into a cathode space (KR) and at least part of the carbon dioxide (CO₂) is reduced to ethylene (C₂H₄) at a cathode (K) and at least part of the ethylene (C₂H₄) is transferred from the catholyte circuit (KK) via the first product outlet (PA1) and the subsequent first connecting conduit (1) into the anolyte circuit (AK).

In some embodiments, bromine (Br₂) is provided in the anode space (AR) and is brought into contact with the ethylene (C₂H₄) transferred into the anolyte circuit (AK) for reaction to form bromohydrin (HOCH₂—CH₂Br) and at least part of the bromohydrin (HOCH₂—CH₂Br) formed is subsequently introduced into a basic environment and dehydrohalogenated therein to form ethylene oxide (C₂H₄O).

In some embodiments, at least part of the bromohydrin (HOCH₂—CH₂Br) formed in the anode space (AR) is introduced into the catholyte circuit (KK) and dehydrohalogenated therein to form ethylene oxide (C₂H₄O).

In some embodiments, a pH below 7 is set in the anode space (AR).

In some embodiments, a pH above 7 is set in the cathode space (KR) or at least in part of the mixing unit.

In some embodiments, at least part of the unutilized and/or reliberated bromine (Br₂) is taken off from the electrolyte mixture and subjected outside the electrolysis cell to a chemical conversion back into a bromide (Br⁻), which bromide (Br⁻) is then added to the electrolyte mixture again.

### BRIEF DESCRIPTION OF THE DRAWINGS

Examples and embodiments of the teachings herein will be described in an illustrative manner with reference to FIGS. 1 to 9 of the appended drawing:

FIG. 1 schematically shows the utilization of oxygen produced in-situ for the epoxidation, according to the teachings of the present invention;

FIG. 2 schematically shows the active pumped circulation of the electrolyte via a diaphragm, according to the teachings of the present invention;

FIGS. 3 and 4 show a comparison of the current yield in ethylene formation from carbon dioxide and from carbon monoxide, according to the teachings of the present invention;

FIG. 5 shows the course of the pH in the electrochemical reduction of carbon dioxide and carbon monoxide to form ethylene in the cathode space, according to the teachings of the present invention;

FIG. 6 shows an electrolysis system for the electrochemical production of ethylene oxide, according to the teachings of the present invention;

FIG. 7 shows an electrolysis system having an external mixing vessel, according to the teachings of the present invention;

FIG. 8 shows an alternative construction of an electrolysis system having an external mixing vessel according to the teachings of the present invention; and

FIG. 9 shows an alternative construction of an electrolysis system having a phase separator according to the teachings of the present invention.

In some embodiments, an electrolysis system for the electrochemical production of ethylene oxide comprises at least one electrolysis cell having an anode in an anode space and a cathode in a cathode space, and also at least one gas separation element, in particular a membrane. The cathode space has a first inlet for carbon monoxide and/or carbon dioxide and is configured to bring this introduced carbon monoxide and/or carbon dioxide into contact with the cathode. The anode space is integrated into an anolyte circuit and the cathode space into a catholyte circuit, with the catholyte circuit having at least one first product outlet for a reduction product.

This first product outlet is connected to a first connecting conduit which is in turn connected to the anolyte circuit. The anode space is configured so that a reduction product which has entered the anode space via this first connecting conduit can be brought into contact with an oxidation product. Here, a reduction product is a substance which is produced electrochemically in the cathodic reduction reaction. The terms anolyte circuit and catholyte circuit refer to hydrodynamic connections, i.e. a conduit system in each case having at least one pump which pumps the electrolyte present in the conduit system, including starting materials, intermediates and products, through the anode space and cathode space. In some embodiments, the cathode reaction and anode reaction are utilized to the same extent, which makes the system very effective. In addition, the system has the advantage that carbon monoxide and/or carbon dioxide can be fed to chemical utilization therein.

Electrolysis systems for the electrochemical utilization of carbon dioxide should also operate ever more efficiently. To ensure a high current density or in experiments seeking to increase this still further, only the carbon dioxide reduction occurring at the catalytically active cathode surface has hitherto been examined. At present, about 80% of the worldwide energy demand is covered by the combustion of fossil fuels, the combustion processes of which result in a worldwide emission of about 34 000 million metric tons of carbon dioxide into the atmosphere per year. The major part of carbon dioxide, which in the case of, for example, a brown coal power station can be up to 50 000 metric tons per day, is disposed of by this liberation into the atmosphere. Carbon dioxide is among the greenhouse gases whose adverse effects on the atmosphere and the climate are being discussed. Since carbon dioxide has a very low thermodynamic position, it can be reduced only with difficulty to form reusable products, but this is made possible by the electrolysis system presented.

A particular advantage here is using carbon monoxide as reducing agent. The formation of ethylene from carbon dioxide (CO₂) always proceeds via the intermediate carbon monoxide (CO):
Step 1: CO₂→CO+½O₂
Step 2: 2CO+2H₂O→C₂H₄+2O₂

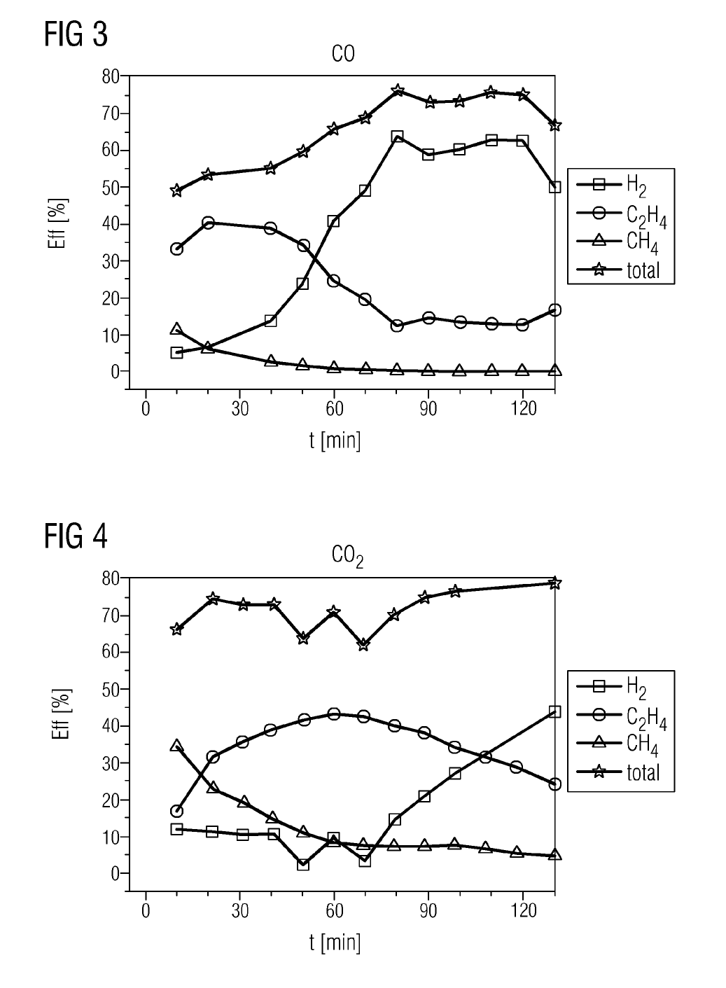

Under the same reaction conditions, the substrates carbon dioxide and carbon monoxide give ethylene in a very similar current yield (see FIGS. 3 and 4). In the rather difficult separation of the ethylene from the catholyte circuit in particular, carbon monoxide as starting material offers an additional advantage: the amount produced and thus the final concentration in the product gas is 50% higher than in the case of carbon dioxide at the same current yield. The reason for this is that twelve electrons have to be transferred in the case of carbon dioxide, while only eight electrons are required for ethylene from carbon monoxide.

The use of carbon monoxide as electrolysis starting material is therefore particularly advantageous when the focus of the reaction is on the production of ethylene oxide. If utilization of carbon dioxide is required, the electrolysis system described makes a combined process for utilization of carbon dioxide and simultaneous production of ethylene oxide possible.

In some embodiments, the gas separation element which separates the anode space and the cathode space from one another is at least one mechanically separating layer, e.g. a separator, a membrane or a diaphragm, which initially separates the electrolysis products formed in the anode space and cathode space from one another. Such an element could also be referred to as separator membrane or dividing layer. Since the electrolysis products are, in some embodiments, gaseous materials, a membrane may have a high bubble point of 10 mbar or greater. The bubble point is a defining parameter for the membrane used which indicates the pressure difference Δp between the two sides of the membrane at which gas flow through the membrane would commence. The membrane can also be a proton- or cation-conducting or -permeable membrane. While molecules, liquids or gases are separated, flow of protons or cations from the anode space to the cathode space or vice versa is ensured. Preference is given to using a membrane which comprises sulfonated polytetrafluoroethylene, e.g. Nafion. Separators based on zirconium oxide, as are used, for example, in electrolyzers which operate under alkaline conditions, are likewise suitable.

In some embodiments, a reduction product can firstly be formed in the catholyte circuit and this can then be combined with further reactants on the anode side or else in an external mixing unit. An electrolysis system having a cell stack made up of a plurality of electrolysis cells can also be provided. In this case, the intermediates would, for example, be transferred into the anode space or cathode space of the next cell. For example, at least one mixing unit which is connected via the first and/or a second connecting conduit to the catholyte circuit and anolyte circuit is provided for this purpose in the electrolysis system. Within this mixing unit, it is possible to use, for example, a vessel having a diaphragm which initially separates the anolyte side and catholyte side further, but, depending on the design of the diaphragm, allows a desired ion exchange between the sides. Active pumping via the diaphragm in the external mixing vessel can also be provided by means of a pumped circuit. The mixing unit can also comprise a product isolation apparatus for ethylene, and also a feed conduit to a gas diffusion anode. Liquid and gas do not necessarily have to be mixed.

In some embodiments, the mixing unit may comprise only one conduit system having a conveying device, e.g. a pump, which brings about mixing within the electrolysis cell. For example, the electrolysis system may comprise a return conduit via which an oxidation product from the anode space and/or an intermediate which is a reaction product of at least one cathodically produced reduction product with an anodically produced oxidation product can be reintroduced into the catholyte circuit. In some embodiments, the intermediate is formed in the anode space and is then supplied to a further reaction in the cathode space, in the catholyte circulation system and/or in the mixing unit.

In some embodiments, the electrolysis system contains bromide ions in its anode space and is configured for oxidizing bromide to bromine at the anode and taking up the reduction product transferred into the anolyte circuit, in particular ethylene, and bringing it into contact with the bromine, which ultimately leads to reaction of the bromine with the ethylene to form the intermediate bromohydrin. In some embodiments, the electrolysis system can also have other halide ions, e.g. iodide or fluoride ions, instead of the bromide ions, and these then form the intermediate by reaction of iodine or fluorine with the reduction product.

In the electrolysis system described, the gas separation element has, for example, a diaphragm or is configured as diaphragm. The main task of the diaphragm is separation of the gases. A diaphragm offers the advantage that, for example, transport of the intermediate, e.g. of a halohydrin, in particular bromohydrin, through the diaphragm from the anode space into the cathode space can occur via a pumped circuit connected to the electrolysis cell. In addition, the diaphragm may be permeable to protons and anions, so that charge equalization between the two cell chambers of the electrolysis cell is ensured.

In some embodiments, the gas separation element comprises a sulfonated polytetrafluoroethylene or is formed by a separator based on zirconium oxide. In such embodiments, the electrolysis cell may be connected to a mixing unit having an external mixing vessel which comprises a diaphragm. The transfer of the intermediate into the catholyte circuit can then be ensured by means of the diaphragm. As sulfonated polytetrafluoroethylene, some embodiments include Nafion which separates molecules, liquids and gases but is permeable to protons and cations and thus again ensures charge equalization within the electrolysis cell.

In some embodiments, the electrolysis system has at least one second product outlet which is configured for taking bromine from an electrolyte mixture conveyed in the anolyte circuit and/or in the catholyte circuit, feeding this into a separate reaction chamber for chemical conversion of the bromine back into a bromide, which reaction chamber is connected via a further connecting conduit to the anode space, so that a hydrodynamic connection between reaction chamber and anode space is established. For the present purposes, an electrolyte mixture is a liquid which comprises, for example, water, one or more different electrolyte salts, electrolysis starting materials, electrolysis products and also intermediates or by-products.

Even when the same electrolyte is used in the anolyte circuit and catholyte circuit or despite at least partial mixing of the circuits, anolyte and catholyte have different proportions of the respective constituents thereof in the anode space and the cathode space. In some embodiments, the reaction chamber and the corresponding connecting conduit can also be configured for converting a different halogen back into its halide. For the chemical conversion of bromine back into a bromide, it is possible to carry out, for example, a further bromination reaction in which hydrogen bromide is formed as downstream product and this can subsequently be fed to a further reaction with potassium hydrogencarbonate or potassium hydroxide, ultimately forming potassium bromide.

This above-described configuration of the electrolysis system with a type of recycling circuit for bromine allows the bromine to be conveyed in a chemical circuit and no additional bromide has to be introduced from the outside but instead this bromide can always be recovered again from the by-product bromine, which means that ultimately no bromide consumption takes place. In some embodiments, the bromine could also be extracted from the electrolysis system and passed to an external further use.

In some embodiments, a reduction product is transferred over to the anode side and there reacted directly, in particular with anodically formed oxygen. For this purpose, the electrolysis system may have an anode which comprises a catalyst and the anode space may contain oxygen. In this case, the anode comprises suitable catalysts and the anolyte comprises at least water from which oxygen can be formed by oxidation at the anode. Suitable catalysts are, for example, manganese, rhenium, platinum, iridium, molybdenum, niobium, and/or silver, but also tungsten-based catalysts or oxides thereof, which may be used in supported form. As catalyst supports, use is made of TiO₂, SiO₂, zeolites such as TS1, MCM 41, SAPO 5, SAPO 34. The anode may comprise a gas diffusion electrode. Furthermore, catalytically active electrode additives comprising activated carbon, carbon blacks, graphites and also binders such as polytetrafluoroethylene or perfluorosulfonic acid and other inert polymers can be present. Use is typically made of anode materials which are inert in respect of the formation of metal halides.

In some embodiments, as an alternative to the in-situ generation of oxygen from the aqueous electrolyte at the anode, there is external introduction of oxygen into the anode space, e.g. via an anode configured as gas diffusion electrode. In the example of this embodiment of the electrolysis system, a further reaction of the reduction product transferred into the anode space with the anodically formed oxygen can then take place. This has the advantage that reduction reactions and oxidation reactions are exploited in both reaction chambers of the electrolysis cell.

In some embodiments, the electrolysis system has at least one suitable catalyst in the cathode space and also suitable reducing agents in an electrolyte environment, which promote selective conversion of the carbon dioxide into ethylene. The ethylene can then be transferred as reduction product into the anode space. In the example described with in-situ oxygen formation at the anode, the ethylene can then be reacted further directly in the anode space to form ethylene oxide, which is an even more valuable chemical than ethylene itself.

In some embodiments, a process for the electrochemical production of ethylene oxide by means of an electrolysis system as described above, includes introducing carbon monoxide and/or carbon dioxide into a cathode space and at least part of the carbon monoxide and/or carbon dioxide are/is reduced therein at a cathode to form ethylene. In addition, at least part of the ethylene is then transferred from the catholyte circuit via a first product outlet and the subsequent first connecting conduit of the electrolysis system into the anolyte circuit. There, an anodic reaction can correspondingly be brought about or carried out so as to produce a valuable chemical and/or an intermediate. The intermediate typically reacts with the ethylene to form a valuable chemical or to form an intermediate, i.e. a further intermediate on the way to a valuable chemical. This production of a valuable chemical occurs simultaneously with the utilization of carbon monoxide and/or carbon dioxide.

In some embodiments, via the intermediate, hereinafter referred to as bromohydrin process, bromine is provided in the anode space and this bromine is supplied with the ethylene transferred into the anolyte circuit to undergo the reaction to form bromohydrin and at least part of the bromohydrin formed is subsequently conveyed into a basic environment and dehydrohalogenated there to form ethylene oxide. No explosive mixing ratios, as can arise, for example, in the direct reaction of ethylene with oxygen, can be formed in the process via the bromohydrin as intermediate. The dehydrohalogenation may be carried out in a basic environment, for which purpose the pH of the catholyte side or the pH of an external dehydrohalogenation chamber is set to and maintained at a value of or greater. Here, the term dehydrohalogenation refers to an elimination reaction in which one hydrogen atom and one halogen atom is split off from the same compound, here specifically usually a dehydrobromination. Here too, the bromine provided can be produced in-situ in the anode space, e.g. from the oxidation reaction in an electrolyte containing bromide ions or the bromine can be provided externally and introduced into the system.

In some embodiments, the process can also occur via a different halohydrin, e.g. an iodohydrin or fluorohydrin. However, the classic chlorohydrin process is unsuitable since it cannot be operated continuously, but instead is dependent on continual introduction of an external base, e.g. calcium hydroxide Ca(OH)₂.

In some embodiments, at least part of the bromohydrin formed in the anode space is fed to the catholyte circuit and dehydrohalogenated there to form ethylene oxide. The ethylene oxide formed thereby can then be separated off from the catholyte circuit. This can, for example, occur by means of a rectification column or by means of a distillation process.

In some embodiments, a pH of 7 or less is set in the anode space in the process. The acidic medium suppresses, for example, the formation of oxygen at the anode and thus ensures that explosive reaction mixtures of oxygen with ethylene are avoided. In addition, the low pH, which can be ensured by means of a buffer in the anode space, ensures that the intermediate halohydrin or bromohydrin is not dehalogenated in the anode space but instead can be conveyed into the cathode space or a suitable external mixing vessel where the end product ethylene oxide can therefore also be taken off.

In addition, a pH of the catholyte in the range from 5 to 11, preferably above 7, is set, for example by means of a buffer solution, in the cathode space or at least in part of the mixing unit or at least in part of the catholyte circuit in the process. The basic environment brings about the dehydrohalogenation of the intermediate bromohydrin.

The bromohydrin process then opens up additional advantages when carbon monoxide is used as substrate: while in the case of the substrate carbon dioxide, the pH always becomes established in the range of the H₂CO₃/HCO₃⁻ buffer of about 7 without external intervention, an additional degree of freedom in the pH arises when carbon monoxide is used as substrate. Eight hydroxide anions (OH⁻) are formed for each ethylene molecule formed from carbon monoxide. However, only one hydroxide anion is consumed by the coupled ethylene oxide formation via bromohydrin. Since hydroxide anions can thus accumulate, a pH which is sufficiently high for this process can be built up and maintained. Although twelve hydroxide anions are formed per ethylene molecule in the case of carbon dioxide, these would be neutralized immediately by further carbon dioxide, which makes the buildup of a high pH impossible.

The use of a carbon monoxide/carbon dioxide mixture allows the pH to be set optimally. The hydrogencarbonate formed is either taken off as material of value when the pH is about 7, or in the case of very high pH values the material of value carbonate is formed as long as appropriately small amounts of carbon dioxide, less than 30%, have been mixed into the carbon monoxide. The above-described mixing of the anolyte and catholyte is likewise possible. Carbon monoxide/carbon dioxide mixtures in the range of 0-100% are possible. In some embodiments, a mixture in the ratio 8:1 is used. If, for example, only traces of carbon dioxide are present in the carbon monoxide, the process can simultaneously be utilized in order to reduce these traces or remove them as hydrogencarbonate (HCO₃⁻) as a result of the basic character of the catholyte. The term traces refers to concentrations of <1%. Concentrations of <0.1%, such as <0.01%, may be employed.

Furthermore, at least part of the unutilized and/or reliberated bromine can, for example, be taken off from the electrolyte mixture in the electrolysis system in the process and subjected outside the electrolysis cell to chemical conversion back into a bromide, which bromide is then introduced again into the electrolyte mixture. For example, a reaction of hydrogen and bromine to form hydrogen bromide and a subsequent reaction with potassium hydrogencarbonate to form potassium bromide can occur. Another example of a utilization of bromine can be a bromination of an aromatic system:

Aromatic+Br₂=Br-aromatic+HBr

The process can thus be supplemented by a bromine-bromide circuit which results in no bromide being consumed and no continuous introduction of bromide into the circuit thus being necessary.

Since mixing of the two circuits is brought about, the bromine can be taken off from the anolyte circuit or from the catholyte circuit or, for example, from the external mixing vessel. However, the recirculation into the system in the form of bromide may occur specifically into the anode space, so that the bromide is concentrated locally at the anode where the bromine circuit can then recommence via the anodic oxidation. The cations additionally bring about charge transport from the anode to the cathode.

In the light of present-day developments in respect of more energy-efficient production processes for basic chemicals and chemical starting materials, electrocatalysis represents a very good possibility for elegant energy conversion. The above-described sustainable synthesis route for producing hydrocarbons is based firstly on the use of a starting material having a low energy content, namely carbon dioxide which is actually a waste product but is used here as carbon source, and secondly on the storage of electric energy in the form of chemical bonds. Here, electric energy which may originate from renewable energy sources or from overcapacities, known as excess energy, can be stored. A further advantage of the combined utilization and production process presented is that product integration into an existing value-added chain of the chemical industry is made possible without new infrastructures having to be created first. The product selection is critically determined by the electrocatalyst used. If the electrocatalytic reduction of carbon dioxide is, by way of example, carried out at copper electrodes, hydrocarbons such as methane or ethylene and also carbon monoxide and hydrogen are mainly formed. The product selectivity is in this case determined, inter alia, by the working electrode potential which can be set.

The annual tonnage production of ethylene is at present 141 million metric tons per annum. Ethylene is a chemically important starting material for many chemicals and materials and is conventionally produced by steam cracking from petroleum or naphtha and then transported via pipelines. The production of the basic chemical ethylene oxide represents a further upgrading of ethylene. At an annular tonnage production of 50 million metric tons per annum, ethylene oxide is used as important key component for the production of substances such as ethylene glycol (55%), polyols (4%), ethanolamines (7%), glycol ethers (12%), surfactants (12%), polyglycols (4%) and others in a lower percentage.

To carry out epoxidation via the bromohydrin process, the cathode space of the electrolysis cell used in the electrolysis system may be configured in such a way that carbon dioxide and/or carbon monoxide is reduced to hydrocarbons, in particular short-chain hydrocarbons such as methane CH₄ or ethylene C₂H₄, over a catalyst. Short-chain hydrocarbons are hydrocarbon compounds CₙHₘ where n<6. For the example of the bromohydrin process, a selective reduction of carbon dioxide and/or carbon monoxide to ethylene C₂H₄ may be carried out. Products such as hydrogen H₂ could, for example, also be produced on the cathode. In some embodiments, a separation device is provided at the cathode space in order to remove by-products from the system. Carbon monoxide CO and bromine Br₂, for example, should not be brought into contact since otherwise Br₂CO is formed and although this can be used in chemical syntheses, owing to its toxicity it should be used in the immediate spatial proximity of the electrolysis plant described.

In some embodiments, the anode space is configured so that the reduction product of the cathodic reaction, in this case the ethylene, can be conducted further to the anode and reacted with the bromine generated in-situ at the anode to form bromohydrin. The process can also be carried out via other halohydrins, but these are somewhat less preferred than the bromohydrin. In particular, the various disadvantages of a chlorohydrin process have been explained above. In some embodiments, the bromohydrin thus generated at the anode is subsequently transferred actively or passively, i.e. via a bypass or via a pumped conduit, directly into the catholyte where it is cathodically dehydrohalogenated in the basic medium thereof.

When using the electrolysis system described, the energy efficiency of the electrolysis system can thus be significantly increased with both half cells being exploited for producing the valuable chemical ethylene oxide from carbon monoxide and/or carbon dioxide. The electrochemical reduction of carbon monoxide and/or carbon dioxide to ethylene and the simultaneous conversion of this ethylene into ethylene oxide in one electrolysis reactor utilizes carbon dioxide, which is important for environmental reasons, but also results in a tremendous economic potential.

Carbon monoxide produced electrochemically from carbon dioxide is not only of interest as substrate or additive. The process also opens up the utilization of various carbon monoxide sources. These are, for example:
coal dust gasification: C+½O₂→CO
smelting gas from steel production
dry reforming of methane CH₄+½O₂→CO+2H₂
reverse water gas shift reaction CO₂+H₂→CO+H₂O
decomposition of formic acid HCOOH→CO+H₂O
decomposition of carbonyls, e.g. Fe(CO)₅→Fe+5CO.

The processes described also tolerate hydrogen in the carbon monoxide fraction in the range from 0 to 80%, preferably from 0 to 20%.

In some embodiments, the further conversion of the cathodically produced ethylene into ethylene oxide can then be carried out, for example, via an intermediate, e.g. via the formation of a halohydrin from cathodically produced ethylene and an anodically produced halogen. The halohydrin is then transported actively back to the cathode where it is dehydrohalogenated in the basic environment of the cathode space and ethylene oxide is formed. The halohydrin can also be fed into a subsequent cell in a cell stack where it is dehydrohalogenated in a basic environment, e.g. in the cathode space, and ethylene oxide is formed. The dehydrohalogenation requires a basic medium having a pH>7, which is brought about locally by the cathode reaction alone.

In some embodiments, to ensure reliable, complete dehydrohalogenation, the pH can be set by means of a buffer to a suitable value. The reaction rate of the dehydrohalogenation is dependent on the bound halogen itself and in the case of iodide occurs more quickly than in the case of bromide, which in turn occurs more quickly than in the case of a chloride, and in the case of a chloride in turn more quickly than in the case of a fluoride. The process is, however, preferably carried out in such a way that an additional introduction of base is avoided and the pH is brought to the appropriate level exclusively by means of the cathodic reduction of water to form OH⁻ ions.

The active transport of the intermediate back to the cathode can, for example, occur via the diaphragm used in the electrolysis cell.

In some embodiments, mixing of anolyte and catholyte can also be carried out in an external vessel where the gaseous products are separated off via a supernatant gas phase and the liquid products remain in the liquid phase. The industrial conversion is based on the use of known membrane/diaphragm electrolyzer technology. A further specific aspect of the electrolysis system presented lies in the configuration of the copper-based gas diffusion electrode and the associated selective reduction of carbon monoxide and/or carbon dioxide to ethylene. One specific aspect lies in the fact that anolyte and catholyte can have the same chemical composition and the two processes profit from the use of a halide, in particular bromide.

The ethylene oxide product can finally be separated off via the gas phase. Separation from the liquid phase is also possible with exploitation of clathrate formation by cooling of the electrolyte in an external crystallizing vessel or in a discontinuously operated mixer-settler apparatus. In some embodiments, a membrane permeation process separates off the product from the electrolysis system.

In some embodiments, oxygen is provided in the anode space, and the ethylene transferred into the anolyte circuit is anodically epoxidized by means of this oxygen to form ethylene oxide. Here, the oxygen provided can be generated in-situ in the anode space, e.g. from the oxidation reaction in an aqueous electrolyte, or the oxygen can be provided externally and introduced into the system. The simultaneous epoxidation of ethylene to ethylene oxide thus upgrades a by-product of the electrochemical utilization of carbon dioxide.

In many purely cathodically operated electrolysis systems based on an aqueous electrolyte, oxygen is always produced as by-product at the anode and this is then released more or less unutilized into the atmosphere. The efficiency of the reduction process for electrochemical ethylene oxide production can be increased tremendously when using the process and electrolysis system described here, as a result of the anodically produced oxygen being utilized simultaneously. The ethylene produced on the cathode side is typically anodically upgraded directly after or simultaneously with the continuing reduction process. This simultaneous, paired utilization of cathode and anode as reaction space which is described here opens up the possibility of formally increasing the Faraday efficiency of the electrolysis system theoretically to 200%.

In the above-described process for the electrochemical production of ethylene oxide by anodic epoxidation by means of oxygen produced in-situ, a copper-based gas diffusion electrode, i.e. a gas diffusion electrode which comprises at least a proportion of copper and accordingly operates ethylene-selectively, may be used on the cathode side. The gas diffusion electrode can, for example, have a carbon fabric or a metal mesh to which the catalyst has been applied. Particularly active ethylene-generating electroreduction catalysts are obtained by the catalyst being deposited in-situ on the cathode. As an alternative to or in addition to the proportion of copper, conductive oxides can also be present as electrically conductive material on the cathode. The catalyst layer of the cathode may have, in order to actively promote ethylene formation, a high wettability by the aqueous electrolyte and also electrical contacting of the catalyst, for example by particles or catalyst sites, and also a possibility of inward and outward diffusion of gaseous starting materials and products, which can be ensured, for example, by the porous design of the cathode.

On the anode side, there may be a silver-based gas diffusion electrode, i.e. a gas diffusion anode having a proportion of silver, at which oxygen is then formed and this reacts further with the ethylene to form ethylene oxide. For this purpose, the anode may have additions of activated carbon, carbon blacks, graphites or other binders, for example polytetrafluoroethylene or sulfonated polytetrafluoroethylene. Other inert polymers can also be used as electrode additives. Apart from silver, transition metal catalysts, for example based on manganese, platinum, iridium, molybdenum, rhenium, niobium, tungsten, are also suitable for the anodic oxygen oxidation, and/or silver or oxides thereof which may be used in supported form. Catalyst supports used are TiO₂, SiO₂, zeolites such as TS1, MCM 41, SAPO 5 or SAPO 34. The electrode may be configured as gas diffusion electrode. The metals can be used as solid material or as mixed metal oxide or, as described above for the cathode side, as supported catalysts, e.g. silver on aluminum oxide. Conductive carbon blacks, e.g. Vulkan XC 72 or acetylene black or Ebonex, metallic particles, and activated carbons may be used to increase the electrical conductivity. In addition, the silver catalyst can once again be deposited in-situ or else ex-situ by electrochemical deposition on a conductive support, e.g. a mesh, metal sheet or woven carbon fiber fabric, in an acidic pH medium. For the electrochemical deposition of the silver catalyst, a pH range from 1 to 4 and also a silver nitrate solution having a concentration in the range from 0.0001 mol to 0.01 mol are preferably recommended. The silver catalyst can also be present in the oxidation states +1 or +2, i.e., for example, as silver(II or I/III) oxide Ag₂O₂ or silver(I) oxide Ag₂O. The number in brackets indicates the oxidation state.

The principle of anodic ethylene epoxidation is thus based on the electrocatalytic reaction of oxygen and ethylene. Here, the oxygen can either be formed in-situ by anodic decomposition of water or the electrode could also be supplied directly with a mixture of oxygen and ethylene in gaseous form. In the case of such external supplying of the oxygen/ethylene mixture to the reaction surface, the composition can be set more precisely and safe handling of the gas mixture which is explosive in some compositions can thus be ensured. This can also be ensured by the oxygen formed in-situ firstly being taken off from the anolyte circuit and then, for example, being introduced in a targeted manner into a second electrolysis cell connected in series. Various recommended operating parameters may be found in table 2.

The electrolysis system for the electrochemical production of ethylene oxide thus may comprise an electrolysis cell having a cathode space and an anode space which may be separated from one another by an ion exchange membrane, typically a proton-conducting ion exchange membrane, to prevent mixing of the electrolytes, especially to prevent mixing of the anodically formed and cathodically formed products.

TABLE 2
Particularly recommended operating parameters
for an anodically operated epoxidation of ethylene.
Parameter:
Gas concentration in % by volume

| C₂H₄ | 14-40 | |
|---|---|---|
| O₂ | 5-9 | |
| CO₂ | 5-15 | |
| Ar (optional) | 5-15 | |
| Temperature | 0-120° | C. |
| Pressure | 1-50 | bar |

If a diaphragm is used, this can comprise, for example, a ceramic or a polymer such as polypropylene, polyethylene, polyvinyl chloride, polytetrafluoroethylene. The use of composite materials in the separation layer between anode space and cathode space is also not ruled out. The carbon monoxide and/or carbon dioxide can be present in dissolved form in the electrolyte or else in gaseous form or can be introduced directly in gaseous form into the process chamber through the cathode configured as gas diffusion electrode. The electrolyte used in the cathode space comprises electrolyte salts, e.g. in a concentration range from 0.1 mol to 3 mol. As electrolyte salts, use is typically made of alkali metal sulfates, alkali metal halides or alkali metal carbonates or alkali metal phosphates. The pH of the catholyte may be set in the range from 5 to 8.

The pH of the electrolyte present in the anode space, on the other hand, may be selected in the basic range, i.e. in a pH range from 7 to 14, which is in any case present as a result of the reaction when oxygen is produced in-situ, but in the case of introduction of oxygen from the outside is set by means of a pH buffer. In the epoxidation by means of anodically produced oxygen in the anode space, e.g. a 0.1-3 molar potassium hydroxide solution or a 0.1 to molar potassium hydrogencarbonate solution. Mixtures of the electrolytes mentioned can also be used. In some embodiments, the addition of the ethylene likewise may be effected directly via the anode as gas diffusion electrode. In this case, the ethylene gas addition rate may be set in the range from 5 to 500 sscm per cm² of electrode area.

Specifically, compared to previously thermally operated catholytic processes for ethylene oxide production, the presented electrochemical epoxidation starting out from carbon monoxide or carbon dioxide has the advantage that the nascent oxygen formed anodically is already present in activated form in the electrolysis system, so that the process can be operated at room temperature. If, for example, a rectification column is used for the isolation of the product, unreacted ethylene, which has a boiling point of −103.7° C., can be separated from the ethylene oxide, which has a boiling point of 10.7° C., and be recirculated to the anode.

In some embodiments, there is an alternative product isolation of the ethylene oxide from the system is the precipitation of clathrates from the anolyte in a temperature range from 2 to 11° C., preferably at 11° C. The ethylene oxide which has been epoxidized in the electrolysis system can be present as a mixture with water at 20° C. The miscibility is directly proportional to the pressure change, corresponding to Henry's Law.

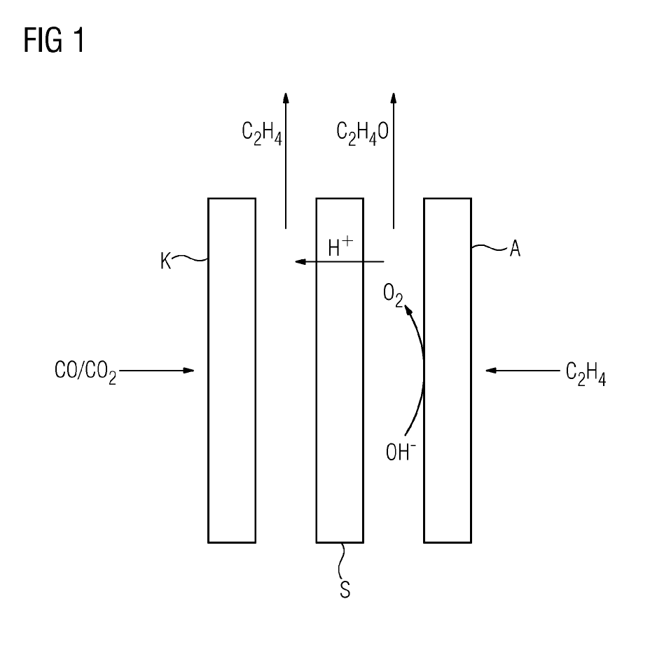

FIG. 1 schematically shows the in-situ production of oxygen O₂ at the anode A, in particular at the anode surface. In FIG. 1, the arrangement of cathode K and anode A on two sides of a separator S is shown in greatly simplified form: on the cathode side, the carbon monoxide CO and/or carbon dioxide CO₂ is introduced and reduced to ethylene C₂H₄. This is brought to the anode side, in this case via an external connecting conduit 1. The separator S is permeable to protons H⁺ in order to ensure charge neutrality in the electrolysis cell EZ. The ethylene C₂H₄ reacts on the anode side directly with anodically produced oxygen O₂ which is produced by oxidation of OH⁻ ions of the electrolyte. As an alternative or in addition, oxygen O₂ can also be introduced from the outside. A reaction of the ethylene C₂H₄ with external oxygen O₂ to form ethylene oxide C₂H₄O can be carried out in the anode space AR or else in a reaction chamber separate from the electrolysis cell EZ.

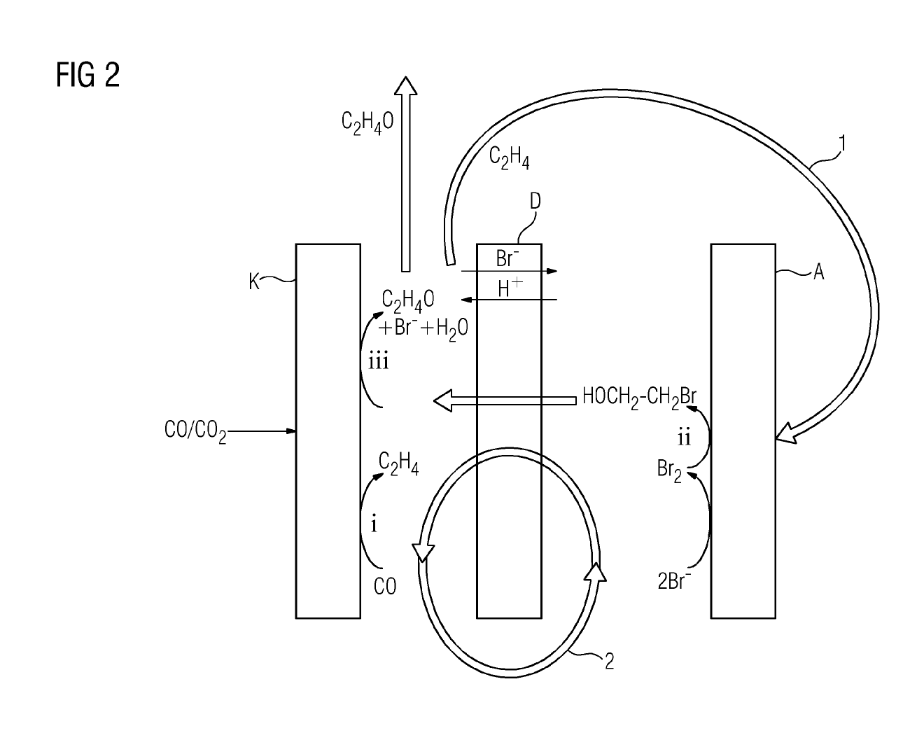

FIG. 2 schematically shows an arrangement of anode A and cathode K which, in particular, indicates the process flow: the reactions proceeding at the anode A are separated off by a diaphragm D from the reactions proceeding in the cathode region. Arrows through the diaphragm D indicate that a certain exchange of ions H⁺, Br⁻ between the two chambers AR, KR of the electrolysis cell EZ occurs. The circular arrow 2 shows the mixing brought about by a mixing unit, e.g. a pump P. The initial electrochemical ethylene oxide production reaction i at the cathode surface, in which carbon monoxide CO and/or carbon dioxide CO₂ is reduced to ethylene C₂H₄, is firstly shown. This ethylene C₂H₄ is first and foremost extracted from the catholyte circuit KK and introduced into the anolyte circuit AK, e.g. via a connecting conduit 1, and brought to the anode A for further reaction. The transfer of the ethylene C₂H₄ to the anode A is again illustrated by a double arrow 1 indicating the flow direction in the schematic depiction of FIG. 2. At the anode A, the further reaction ii, for example, of the ethylene C₂H₄ to form an intermediate Int then takes place: bromide ions Br⁻ present in the electrolyte are oxidized at the anode A to form bromine Br₂ which then reacts further with the ethylene C₂H₄ to form bromohydrin HOCH₂—CH₂Br. This is, for example, transported by means of the mixing unit 2 through the diaphragm D back to the other side of the electrolysis cell. Once again at the cathode K, the reaction iii to form ethylene oxide C₂H₄O, water H₂O and bromide Br⁻ takes place due to a lower pH than on the anode side.

FIG. 2 additionally shows that bromide ions Br⁻ and protons H⁺ can pass through the diaphragm D to effect charge equalization. A further double arrow shows the offtake of ethylene oxide C₂H₄O from the catholyte surface KK. Cathode space KR and anode space AR of the electrolysis cell EZ may be separated by a diaphragm D which at least prevents mixing of the gases. Such a diaphragm D can consist of a ceramic or of a polymer, for example polypropylene, polyethylene, polyvinyl chloride, and/or polytetrafluoroethylene. The use of fiber-reinforced composites, e.g. zirconium oxide ZrO₂ or zirconium phosphate Zr₃(PO₄)₄ in a polymer matrix, in the diaphragm D is also not ruled out.

FIGS. 3 and 4 show a comparison of the Faraday efficiencies Eff in the formation of ethylene C₂H₄ from carbon dioxide CO₂ and carbon monoxide CO. It can be shown experimentally that the formation of ethylene C₂H₄ proceeds via the intermediate carbon monoxide CO. Under the same reaction conditions, the substrates carbon dioxide CO₂ and carbon monoxide CO give ethylene C₂H₄ in a very similar current yield Eff. The current yields Eff for hydrogen H₂ and methane CH₄ are also shown.

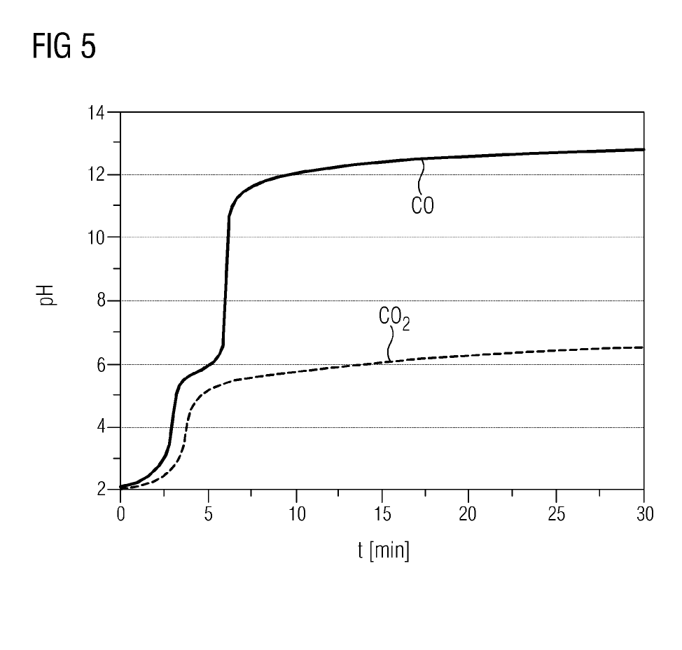

FIG. 5 shows the course of the pH in the electrochemical reduction of carbon dioxide CO₂ and carbon monoxide CO to ethylene C₂H₄ in the cathode space KR. If carbon monoxide CO is used instead of carbon dioxide CO₂ as substrate in the bromohydrin process, the pH profile in the system also changes.

In the reaction of carbon dioxide CO₂, the pH always becomes established in the range of the H₂CO₃/HCO₃⁻ buffer at about 7 without external intervention. When carbon monoxide CO is used as substrate, an additional degree of freedom in the pH is obtained: eight hydroxide anions OH⁻ are formed for each molecule of ethylene C₂H₄ formed from carbon monoxide CO. However, only one hydroxide anion OH⁻ is consumed by the coupled formation of ethylene oxide via bromohydrin. Since hydroxide anions OH⁻ can thus accumulate, a pH which is sufficiently high for this process can be built up and maintained. Although 12 hydroxide anions OH⁻ are formed per molecule of ethylene C₂H₄ in the case of carbon dioxide, these would immediately be neutralized by further carbon dioxide CO₂, which makes the buildup of a high pH impossible. The system can therefore be utilized, for example, for producing potassium hydroxide KOH. For constant long-term operation, the electrolyte is appropriately worked up. Apart from bromine or Br₂CO, the plant can also produce further building blocks for syntheses.

The pH can be set by means of a carbon monoxide/carbon dioxide mixture. The hydrogencarbonate formed is either taken off as material of value when the pH is about 7, or in the case of very high pH values, above 11, the material of value carbonate is formed if appropriately small amounts of carbon dioxide below 30% are mixed into the carbon monoxide CO, e.g. by mixing of the anolyte and catholyte. Carbon monoxide/carbon dioxide mixtures having a proportion of carbon dioxide of from 0.01% to 30% are particularly suitable. Apart from potassium hydroxide KOH, potassium carbonate K₂CO₃ could also be produced by means of the process.

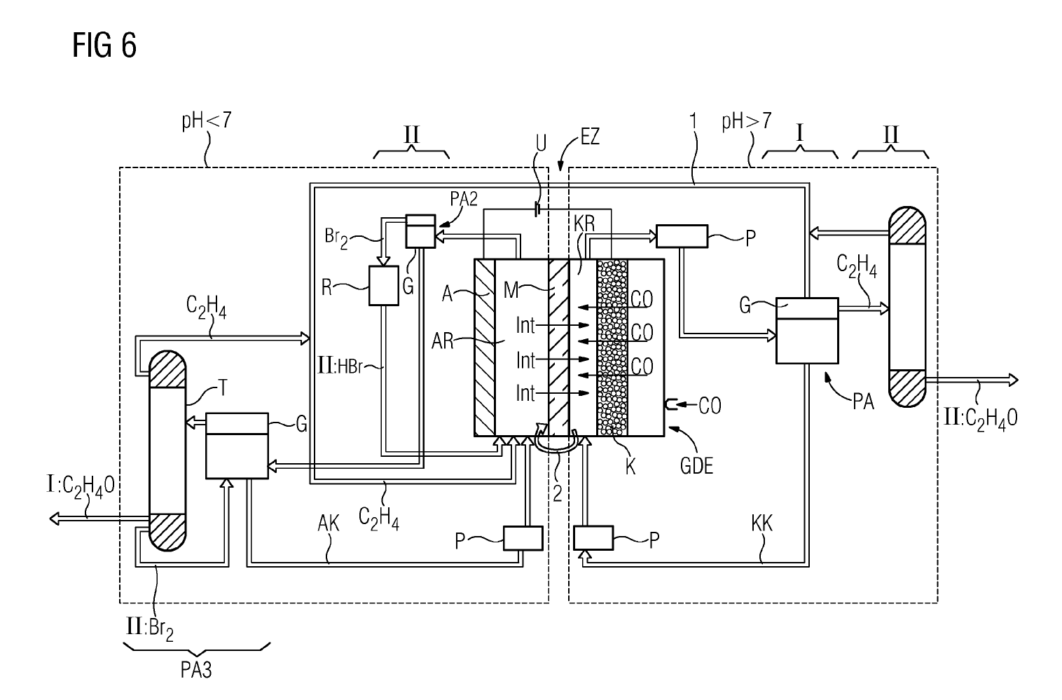

FIG. 6 schematically shows an electrolysis plant as can be used for the electrochemical production of ethylene oxide. Even though the anolyte circuit AK and the catholyte circuit KK, as described in the present patent application, are connected to one another, the anolyte side and the catholyte side are indicated by two regions of the electrolysis system enclosed by broken lines in the schematic depiction. In particular, the depiction clearly shows that, despite hydrodynamic connections 1, 2 between the two circuits AK, KK, a local pH difference distinguishes the anolyte circuit AK and the catholyte circuit KK. The electrolysis cell EZ has an anode A in an anode space AR and a cathode K in a cathode space KR, with anode space AR and cathode space KR being separated from one another and joined to one another by a membrane M. The cathode K can, for example, be a gas diffusion electrode GDE via which the carbon monoxide CO and/or carbon dioxide CO₂ can be introduced into the catholyte circuit KK. Both circulation systems AK, KK are preferably provided, as shown in FIG. 1, with pumps P which serve to effect the necessary circulation of the mixture of electrolytes, starting materials and products through the electrolysis system. Anode A and cathode K are electrically connected to one another via a voltage supply U and via the electrolyte.

The catholyte circuit KK may include at least one product outlet PA1, here shown by way of example as gas separation chamber G, via which at least one cathodically produced electrolysis product, in particular ethylene C₂H₄, can be taken off from the catholyte circuit KK. This is then fed via a connecting conduit 1 into the anode space AR. The anolyte circuit AK is also provided with at least one product outlet PA3, which once again can comprise a gas separation chamber G as shown in FIG. 6, via which anodically produced ethylene oxide C₂H₄O can be taken off from the anolyte circuit AK. This product outlet PA3 in the anolyte circuit AK can in case I serve to effect extraction of anodically produced ethylene oxide C₂H₄O from the anolyte circuit. In case II, in which an intermediate Int and ethylene oxide C₂H₄O produced on the cathode side are employed, this product outlet PA3 in the anolyte circuit AK can be used for the removal of unreacted ethylene oxide C₂H₄O or for recirculation of bromine Br₂, Br⁻ into the circuit. In the variant II via a halohydrin as intermediate Int, this can, for example, be transferred directly via the membrane M from the anode space AR into the cathode space KR, typically aided by active mixing, which is indicated by an arrow 2 in FIG. 6. Different constructions of electrolysis cells EZ having a mixing unit Mi are shown in FIGS. 7 to 9.

In the case of the variant II, in which the ethylene oxide C₂H₄O is produced via an intermediate Int, an additional product outlet PA2 is preferably provided in the anolyte circuit AK. This product outlet PA2 can firstly take off the bromine Br₂ from the anolyte circuit AK, once again by means of a gas separation device G. The bromine Br₂ can then be introduced into a reaction chamber R where, for example, a bromination to form HBr and a subsequent further reaction, e.g. via potassium hydrogencarbonate KHCO₃ to form potassium bromide KBr, is carried out, so that bromide Br⁻ can again be introduced in the form of potassium bromide KBr back into the anolyte circuit AK and thus into the overall electrolysis circuit. Thus, bromine Br₂ neither has to be discharged nor has to be processed in another way or stored. In addition, the electrolysis plant in this way operates without consumption of bromide. Potassium hydrogencarbonate KHCO₃ is a further material of value which is not consumed and therefore does not have to be introduced in extra amounts but instead is formed as by-product of the electrolysis and thus assists a closed circuit.

In the process, the carbon monoxide CO and/or carbon dioxide CO₂ is largely present in dissolved form in the electrolyte, but the carbon monoxide CO and/or carbon dioxide CO₂ can also be present in gaseous form or in chemically bound form in the circuit. In gaseous form, it can, for example, be introduced directly through the cathode K into the process chamber KR when a gas diffusion electrode GDE is used.

The ethylene C₂H₄ may be introduced via a gas diffusion electrode GDE, in this case via the anode A, into the electrolysis system. The ethylene gas introduction rate is preferably selected in the range from 5 to 500 sscm per cm² of electrode area; sscm is a measure of the flow rate: cm³ per second based on standard conditions (0° C., 101 kPa).

Finally, the use of a membrane M in the electrolysis cell EZ or in the external mixing vessel Mi and/or the use of a rectification column T, by means of which unreacted ethylene C₂H₄ can be separated from the ethylene oxide C₂H₄O is recommended for the removal or separation of product from the electrolysis circuit. Ethylene C₂H₄ and ethylene oxide C₂H₄O have very different boiling points: ethylene C₂H₄ boils at −103.7° C., i.e. is gaseous at room temperature, while ethylene oxide C₂H₄O has a boiling point of 10.7° C. The unreacted ethylene C₂H₄ can in this way be recirculated to the anode A.

In some embodiments, separating off the ethylene oxide C₂H₄O product includes precipitation of clathrates (clathrate hydrates) from the anolyte. This is carried out in a temperature range from 2 to 11° C., preferably at 11° C. The clathrates contain up to 26% by weight of ethylene C₂H₄, which corresponds to 46 water molecules and 6.66 ethylene molecules per unit cell. The clathrates can then be separated off and the ethylene C₂H₄ can be liberated again thermally, e.g. in a temperature range from 11 to 200° C. In some embodiments, minor amounts of ethylene glycol to be formed by hydrolysis in the liquid phase. In the case of a 3% strength mixture with water at a pH in the range from 5 to 9, these attain a half life of about 20 days.

Various working examples for the mixing process are shown in FIGS. 7 to 9: in the bromohydrin process presented, the cathodic reduction of carbon monoxide and/or carbon dioxide to ethylene C₂H₄ is thus combined with the simultaneous anodic formation of bromohydrin HOCH₂—CH₂Br from the ethylene C₂H₄ and anodically produced bromine Br₂. The basis of this process is therefore the anodic oxidation of bromide Br⁻ to bromine Br₂. As an alternative, the process presented can also be operated as halohydrin process, i.e. the further halogens can be employed as alternatives to bromine Br₂.

The bromohydrin HOCH₂—CH₂Br formed on the anode side AR is then actively pumped to the cathode side KR in the process. Mixing can be carried out either continuously or discontinuously. For example, the anolyte can, as shown in FIG. 2, be circulated via a diaphragm D. Here, for example, a bypass in which a pump promotes mixing can be provided between the anode space AR and cathode space KR. As an alternative, it is possible, as shown in FIGS. 7 to 9, for mixing of anolyte and catholyte to be carried out in an external mixing vessel Mi. For pumping through a diaphragm D, the latter may be porous.

FIG. 7 shows an embodiment having a mixing vessel Mi in which a diaphragm D and/or a bypass and a pump P are again provided. In this example, the electrolysis cell EZ preferably has a membrane M. This membrane M is preferably composed of sulfonated polytetrafluoroethylene (PTFS), which is usually known as Nafion. The mixing vessel Mi has a first section Mi1 which is hydrodynamically connected, e.g. via a tube, to the anode space AR and a second section Mi2 which is hydrodynamically connected, e.g. via a second tube, to the cathode space KR. The two sections of the mixing vessel Mi are joined to one another by a diaphragm D via which mixing of anolyte and catholyte can occur. In this variant of the electrolysis system, the cathode space KR preferably has at least one inlet GDE for carbon monoxide and/or carbon dioxide CO₂ and the anode space AR preferably has at least one inlet for ethylene C₂H₄. As electrolyte, preference is given to using an alkali metal bromide, e.g. potassium bromide in aqueous solution KBr (aq): the bromide Br⁻ can then be oxidized to bromine Br₂ in the anode space AR. The bromine Br₂ can, for example, then be taken off from the system via the first region Mi1 of the mixing vessel Mi and is typically, as described above, fed back as bromide Br⁻ into the system. The ethylene C₂H₄ and also the end product ethylene oxide C₂H₄O can be extracted from the second region Mi2 of the mixing vessel Mi.

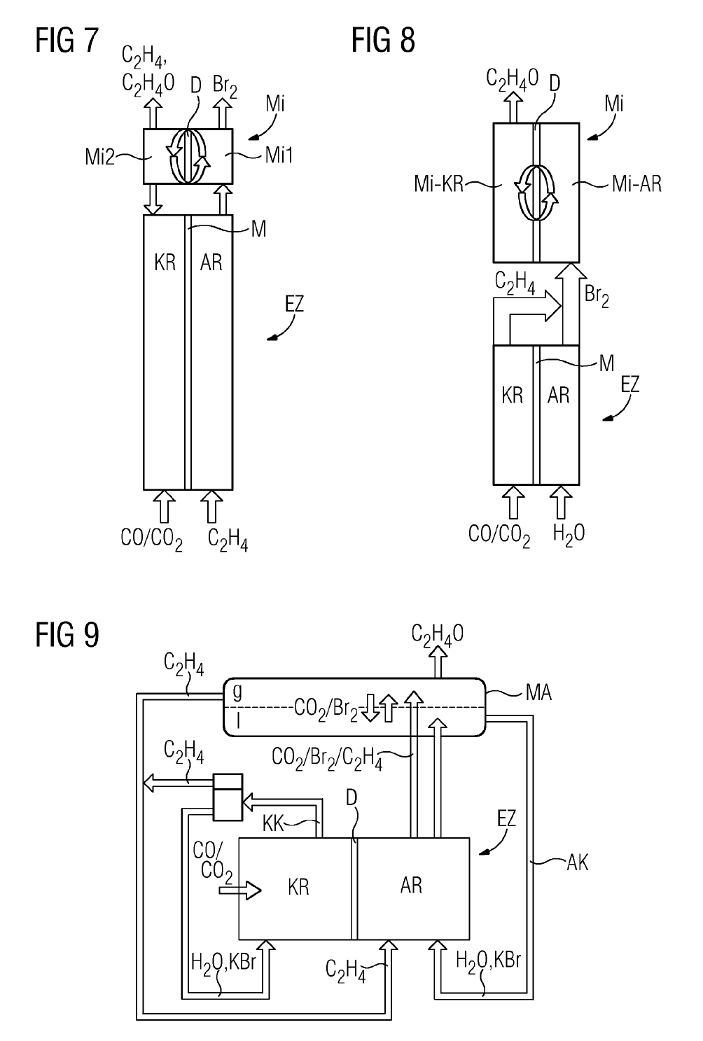

In a manner similar to the variant in FIG. 7, the electrolysis system shown in FIG. 8 is provided with an electrolysis cell EZ and an external mixing vessel Mi. Once again, the electrolysis cell EZ has a membrane M, in particular a Nafion membrane, and the mixing vessel Mi-E has a diaphragm D for separating anode side and cathode side or anolyte and catholyte. As a distinction from the construction in FIG. 7, the products from the cathodic reduction process and the anodic oxidation process are taken off from the electrolysis cell EZ and combined before they flow into the first region Mi-AR of the mixing vessel Mi-E. An exchange between the first chamber Mi-AR of the mixing vessel Mi-E and the second chamber Mi-KR of the mixing vessel Mi-E can occur through the diaphragm D in the mixing vessel Mi-E. This exchange can once again be driven by a pump, e.g. by means of a bypass system having a pump. In the second section Mi-KR of the mixing vessel Mi-E, the further reaction of the bromohydrin HOCH₂—CH₂Br to form ethylene oxide C₂H₄O then occurs.

The mixing vessel Mi-E is in this case connected as second electrolysis cell Mi-E downstream of the first electrolysis cell EZ. The electrolysis products from the first electrolysis cell EZ are introduced into the anode space Mi-AR of the mixing electrolysis cell Mi-E. There, the production of bromohydrin HOCH₂⁻ CH₂Br occurs in the anode space Mi-AR of the mixing cell Mi-E simultaneously with the reduction of carbon monoxide and/or carbon dioxide, and this bromohydrin then goes through the diaphragm D into the cathode space Mi-KR in which a pH above 7 prevails, promoting the further reaction to form ethylene oxide C₂H₄O. The pH can, for example, be set by means of a buffer.

The intermediate gases ethylene C₂H₄ and bromine Br₂ may be taken off from the anolyte and catholyte circuits of the first electrolysis cell system EZ and introduced into the anolyte of the active mixing cell Mi-E.

Finally, FIG. 9 shows an illustrative construction having an external mixing vessel MA which uses a phase separator construction MA: the electrolysis cell EZ is again made up of a cathode space KR and an anode space AR which are joined to one another via a diaphragm D. An inlet for carbon monoxide CO and/or carbon dioxide CO₂ and also an inlet for the electrolyte (starting material) mixture are provided in the cathode space KR. An aqueous potassium bromide solution KBr (aq) may be used as electrolyte. The same electrolyte basis is typically used in the mixing process on both sides, i.e. as anolyte and as catholyte. At least one inlet for ethylene C₂H₄, which in the process described is formed in the cathode space KR and is correspondingly taken off from the catholyte circuit KK, is then provided in the anode space AR. The bromine-bromide circuit may be closed in a manner comparable to FIG. 1.

In the example of FIG. 9, the anolyte circuit AK has a phase separator MA. As phase separator, there may be a mixer-separator MA into which carbon monoxide CO and/or carbon dioxide CO₂, bromine Br₂, and ethylene C₂H₄ go and are present in gaseous form in the upper volume section g of the mixer-separator MA. From this volume g, the ethylene C₂H₄ is then, for example, conveyed further back into the electrolyte circuit. The carbon monoxide CO and/or carbon dioxide CO₂ which has not been converted and also the bromine Br₂ are in an electrolyte equilibrium, both in the gaseous phase g and in the liquid phase 1 of the electrolyte mixture, see table 3.

TABLE 3
The electrolyte equilibria in bromine-containing
water differ as a function of the pH range

| Acidic pH | Basic pH |
|---|---|
| Br₂ + H₂O [symbol] H⁺ + Br⁻ + HOBr | Br₂ + OH⁻ [symbol] Br⁻ + OBr⁻ + H₂O |
| HOBr [symbol] H⁺ + OBr⁻ | HOBr + OH⁻ [symbol] OBr⁻ + H₂O |
| Br₂ + Br⁻ [symbol] Br₃⁻ | Br₂ + Br⁻ [symbol] Br₃⁻ |
| | 3 OBr⁻ [symbol] 2 Br⁻ + BrO₃⁻ |

The end product ethylene oxide C₂H₄O can be taken off from the gas volume g in the mixer-separator MA. A retention device for gases which are not wanted in the end product, e.g. bromine Br₂, is preferably provided at the product outlet. This retention device can, for example, be pH-dependent. The effective removal of bromine is important since bromine at the cathode would be bad for the efficiency. The mixer-separator MA, too, can once again have a diaphragm D. This should in any event prevent gaseous reactants and products from mixing. The mixing apparatus MA may be used for conveying the bromohydrin HOCH₂—CH₂Br produced on the anode side AR or a different halohydrin to the cathode side KR where it is dehydrohalogenated in the basic catholyte and ethylene oxide C₂H₄O is ultimately formed in this way.

The process described is not restricted to ethylene C₂H₄ and ethylene oxide C₂H₄O. Extension to other olefins and olefin oxides is also possible.

The above-described electrolysis systems and the process for ethylene oxide production has the advantage that economically useful products are produced at both the anode A and the cathode K. The efficiency of the overall process or of the system is increased by the combination of the two cell reactions.

The anode A may comprise a tantalum or platinum anode and/or a gas diffusion electrode. Anode materials which are inert in respect of the formation of metal halides are typically used. As electrode additives, it is possible to use activated carbons, carbon blacks and graphites, and polytetrafluoroethylene PTFE, perfluorosulfonic acid PFSA, and other inert polymers as binders.

In some embodiments, cathode K comprises copper-based gas diffusion electrodes GDE which contain an ethylene-selective catalyst. The gas diffusion electrode GDE can consist of a woven carbon fiber fabric or a metal mesh onto which the catalyst has been applied. Particularly active ethylene-generating electroreduction catalysts are obtained by depositing the catalyst in-situ on the cathode K. However, ex-situ deposition of the catalyst on the cathode fabric or mesh is also conceivable. The substrate does not necessarily have to be a copper substrate or copper-containing substrate. Any conductive material, in particular also conductive oxides, can be employed as substrate for the gas diffusion electrode GDE. The gas diffusion electrode GDE which is used can be realized by means of the porous configuration of the cathode K.

For the selective formation of ethylene at the cathode K, the catalyst layer may satisfy the following criteria: it is wettable by aqueous electrolytes, the catalyst can be electrically contacted, in particular when it consists of catalyst particles or catalyst sites, and the diffusion of gaseous starting materials and products to or from the catalyst can occur unhindered.

Transition metal catalysts based on molybdenum, iridium, platinum, palladium, tungsten, rhenium, rhodium, or alloys of these elements can also be used as catalyst in the cathode K. Here, the metals can be used as solid material or as mixed metal oxide, e.g. as supported catalysts. One method of producing the catalyst occurs via electrochemical deposition on a conductive support which is, in particular, a mesh, metal sheet or woven carbon fiber fabric. The electrochemical deposition of the catalyst may be carried out in-situ in an acidic pH environment. In some embodiments, the electrochemical deposition of the catalyst may be carried out in a pH range from 1 to 4.

In the above-described electrolysis system and process for the electrochemical conversion of carbon monoxide CO and/or carbon dioxide CO₂ into ethylene oxide C₂H₄O, particular attention has to be paid to certain operating parameters:

As indicated in FIG. 6, in some embodiments, an acidic environment, i.e. a pH of <7, prevails in the anode region, and a basic environment, i.e. a pH of >7, prevails in the cathode region. This pH can, for example, be buffered by the carbon dioxide. If only a low oxygen overpotential prevails in the process, some formation of oxygen O₂ occurs at the anode A. However, the formation of oxygen O₂ should be avoided in the halohydrin process since, firstly, a higher overpotential than for the formation of the corresponding bromine gas Br₂ or other halogen is required, and secondly because explosive mixtures with ethylene C₂H₄ can be formed. Accordingly, the formation of oxygen O₂ should preferably be avoided in the bromohydrin process, for example by use of suitable electrolytes and electrode materials. A reaction scheme with anodic dissociation of water and corresponding production of oxygen is shown below. At the cathode K, the following reactions would firstly proceed:

12CO₂+72H⁺+72 e⁻→6C₂H₄+24H₂O

6HOCH₂—CH₂Br+6OH⁺→6C₂H₄O 6Br⁻+6H₂O

6H₂O+6 e⁻→6H⁺+6OH⁻+6e⁻

At the the anode A, the following reactions would accordingly proceed:

12H⁺+12Br⁻→6Br₂+12 e⁻+12 H⁺

6Br₂+6C₂H₄→6H₂C—CH₂Br⁺+Br⁻

6H₂C—CH₂Br⁺+6H₂O→6HOCH₂—CH₂Br+6H+

30H₂O→15O₂+60H⁺+60 e⁻

Overall, carbon monoxide CO and/or carbon dioxide CO₂ and water H₂O are thus converted into oxygen O₂ and ethylene oxide C₂H₄O in this set of reactions:

12CO₂+12H₂O→6C₂H₄O+15O₂

When using an ethylene-selective catalyst material in the cathode space KR and an appropriately highly concentrated bromine-containing electrolyte solution, the oxygen overpotential of the system is increased correspondingly and no oxygen O₂ is formed. The following reaction scheme can thus proceed in the abovementioned electrolysis via the bromohydrin intermediate.

At the anode A, the following reactions then occur:

72H⁺+72Br⁻→36Br₂+72 e⁻+72H⁺

6Br₂+6C₂H₄→6H₂C—CH₂Br⁺+6Br⁻

6H₂C—CH₂Br⁺+6H₂O→6HOCH₂—CH₂Br+6H⁺

Overall, bromine Br₂ and also ethylene oxide C₂H₄O are thus formed in an anodic bromide oxidation in the system:

12CO₂+60HBr→6C₂H₄O+30Br₂+18H₂O

Depending on the pH-dependence of the halogen produced, various ions of the halide can also be produced. For example, OBr⁻ or BrO₃⁻, which are present in a type of electrolyte equilibrium, can be produced as a function of the prevailing pH, see also equations in table 3. Such bromides, hypobromites, bromites or bromates can also occur as corresponding acid-base pairs or as alkali metal salts.

The electrolyte present in the cathode space KR may be selected with concentrations in the range from 0.1 M to 3 M. As electrolyte salts, preference is given to using alkali metal halides, alkali metal carbonates or alkali metal phosphates. The pH of the catholyte may be set, at least locally in the cathode space KR, to a value in the range from 5 to 11. The pH of the electrolyte in the anode space AR, on the other hand, may be set so as to be slightly acidic, i.e. in any case below 7, or below 5, in order to suppress the formation of oxygen O₂. The electrolyte present in the anode space AR is typically the same as in the catholyte circuit KK and accordingly has a concentration in the range from 0.1 M to 3 M. In some embodiments, mixtures of the electrolyte have a concentration of from 0.1 M to 3 M of a metal halide, e.g. potassium bromide KBr, potassium chloride KCl or potassium iodide KI, or have an addition of a carbonate, e.g. potassium hydrogencarbonate KHCO₃, in a concentration of from 0.1 M to 1 M. The use of a three molar potassium bromide solution KBr (aq) has been found to be particularly useful, and this may be used as anolyte and also as catholyte. Any electrolytes containing bromide Br⁻ can lead to the formation of bromohydrins HOCH₂—CH₂Br, which can be dehydrohalogenated relatively easily, particularly compared to chlorine compounds. Mixtures of the electrolytes proposed can also be used.

The process described and the electrolysis system described offer the advantage over the previously known electrochemical processes that maximum exploitation of the introduced electric energy by the utilization of materials in the cathode reaction and the anode reaction is made possible. A further advantage is that exclusively water H₂O and carbon monoxide CO and/or carbon dioxide CO₂ are required as reactants, i.e. as starting materials. In contrast to the processes known hitherto, no hydrogen H₂ is formed as unutilized waste product.

In contrast to all processes known hitherto, the active addition of carbon dioxide makes it possible for hydrogencarbonate to be formed according to the following reaction equilibrium while at the same time avoiding the formation of 1,2-dichloroethane:

CO₂+H₂O≈H₂CO₃≈HCO₂⁻+H⁺≈CO₃²⁻+2H⁺

The bromohydrin process has the further advantage that the bromide or another halide used has a positive effect on the formation of ethylene C₂H₄, since the hydrogen overpotential at the cathode K is increased by the presence of a halide in the electrolyte. In addition, the process presented tolerates, especially as a result of the gas diffusion electrodes GDE used, higher temperatures than the processes known hitherto in the literature. Furthermore, it is a great advantage that the formation of waste products, for example calcium chloride which is formed, for example, in the chlorohydrin process, is avoided by the epoxidation of ethylene C₂H₄ in the bromohydrin process described.

Compared to known thermal catalysis processes, the epoxidation described here has the advantage of suppressing undesirable total oxidation of ethylene C₂H₄. As a result, no hot spots as arise in thermally operated gas-phase processes, e.g. in hollow spaces in the catalyst bed, occur. In the thermally operated gas-phase processes, undesirable formation of carboxylic acids, which then lead to degradation of the catalyst, can occur. Degradation effects such as blocking of the catalyst pores as a result of abrasion, poisoning of the catalyst by sulfur, corrosion or changes in the particle morphology, e.g. due to agglomerate formation, are also avoided in the process described. The electrochemical process described basically avoids any thermally induced aging effects.

What is claimed is:

1. An electrolysis system for the electrochemical production of ethylene oxide, the system comprising:
an electrolysis cell having an anode in an anode space and, a cathode in a cathode space;
a gas separation element;
wherein the cathode space has a first inlet for carbon monoxide and/or carbon dioxide and is configured for bringing the introduced carbon monoxide and/or carbon dioxide into contact with the cathode;
the anode space is integrated into an anolyte circuit and the cathode space is integrated into a catholyte circuit;
the catholyte circuit has a first product outlet for a reduction product joined to a first connecting conduit connected to the anolyte circuit; and
the anode space is configured for bringing a reduction product introduced via the first connecting conduit into contact with an oxidation product.

2. The electrolysis system as claimed in claim 1, further comprising a mixing unit hydrodynamically connected to the anolyte circuit and the catholyte circuit.

3. The electrolysis system as claimed in claim 1, wherein the anode space contains bromide ions and is configured for oxidizing bromide to bromine and for taking up a reduction product transferred into the anolyte circuit and bringing it into contact with the bromine.

4. The electrolysis system as claimed in claim 1, wherein the gas separation element comprises a diaphragm.

5. The electrolysis system as claimed in claim 1, wherein the gas separation element (M) comprises a sulfonated polytetrafluoroethylene.

6. The electrolysis system as claimed in claim 1, further comprising:
a second product outlet configured for taking bromine off from an electrolyte mixture conveyed in the anolyte circuit and/or catholyte circuit; and
a separate reaction chamber for chemical conversion back into a bromide;
wherein the reaction chamber is connected hydrodynamically via a further connecting conduit to the anode space.

7. A method for the electrochemical production of ethylene oxide by means of an electrolysis system, the method comprising:
introducing
carbon monoxide (C) and/or carbon dioxide into a cathode space;
reducing at least part of the carbon dioxide to ethylene at a cathode; and
transferring at least part of the ethylene from the catholyte circuit via a first product outlet and a subsequent first connecting conduit into an anolyte circuit.

8. The method as claimed in claim 7, further comprising:
providing bromine in the anode space;
combining the bromine with the ethylene transferred into the anolyte circuit for a reaction to form bromohydrin; and
subsequently introducing at least part of the bromohydrin formed into a basic environment and dehydrohalogenating the bromohydrin therein to form ethylene oxide.

9. The method as claimed in claim 8, further comprising:
introducing at least part of the bromohydrin formed in the anode space into the catholyte circuit; and
dehydrohalogenating the bromohydrin therein to form ethylene oxide.

10. The method as claimed in claim 7, further comprising setting the anode space to a pH below 7.

11. The method as claimed in claim 7, further comprising setting a pH above 7 in the cathode space or in at least part of the mixing unit.

12. The method as claimed in claim 7, further comprising:
taking off at least part of the unutilized and/or reliberated bromine from the electrolyte mixture;
converting the bromine outside the electrolysis cell back into a bromide; and
adding the bromide to the electrolyte mixture again.
